(19) United States
(12) Patent Application Publication
(10) Pub. No.: US 2019/0209526 A1
(43) Pub. Date: Jul. 11, 2019

(54) Stable Atorvastatin Suspension Composition

(71) Applicant: Rosemont Pharmaceuticals Limited, Leeds, Yorkshire (GB)

(72) Inventors: Paul Hutchinson, Leeds, Yorkshire (GB); Tajamal Mustafa, Huddersfield, Yorkshire (GB)

(21) Appl. No.: 16/325,438

(22) PCT Filed: Aug. 10, 2017
(86) PCT No.: PCT/GB2017/052366

§ 371 Date: Feb. 14, 2019

Related U.S. Application Data
(60) Provisional application No. 62/376,158, filed on Aug. 17, 2016.

(51) Int. Cl. A61K 31/4015; A61K 47/14; A61K 47/02; A61K 9/00; A61K 9/10; A61K 47/26

(52) U.S. Cl. CPC A61K 31/4015; A61K 47/14; A61K 47/26; A61K 9/0053; A61K 9/10; A61K 47/02

(57) ABSTRACT

The invention relates to a composition atorvastatin, or a pharmaceutically acceptable salt, solvate or hydrate thereof, and a non-polar carrier, wherein said composition is in the form of a suspension.

### BACKGROUND OF THE INVENTION

Statins are a class of drugs that inhibit the enzyme HMG-CoA reductase, which plays a central role in the production of cholesterol. They are used for lowering cholesterol levels in individuals and for the prevention and management of cardiovascular diseases. Atorvastatin is a member of the statins drug class.

Statins are widely available in the form of tablets for oral administration. Some individuals, however, have difficulty or are unable to take solid oral pharmaceutical dosage forms. These individuals include, for example, infants, elders, and others suffering from dysphagia. There is thus a need for stable oral liquid statin formulations, and specifically, atorvastatin formulations.

WO2013088161 describes pharmaceutical transmucosal statin compositions. The compositions of WO2013088161 are either in the form of a solution (wherein lipophilic statins are solubilized in an oil, or hydrophilic statins are solubilized in water), or in the form of a suspension or emulsion (wherein lipophilic statins are suspended or emulsified in water, or hydrophilic statins are suspended or emulsified in an oil).

### DESCRIPTION OF THE INVENTION

The present invention provides statin compositions. Specifically, the invention provides compositions which comprise atorvastatin, or a pharmaceutically acceptable salt, solvate or hydrate thereof, and at least one non-polar carrier, which preferably comprises glyceride, and more preferably triglyceride.

Hereinafter, the term ‘atorvastatin’ is used to describe any of atorvastatin, a pharmaceutically acceptable salt, solvate or hydrate thereof.

In an embodiment of the invention, the composition is in the form of a suspension, in which solid atorvastatin particles are suspended in the carrier. The term ‘suspension’ refers to a heterogeneous mixture comprising solid particles which are dispersed in a liquid phase.

Surprisingly, it has been found that the atorvastatin compositions of the present invention are highly stable as compared to aqueous atorvastatin compositions. After standing for 3 months at an elevated temperature, the compositions of the invention remained stable and contained substantially less degradation products as compared to an analog aqueous atorvastatin suspension composition.

The compositions of the invention comprise, as an active ingredient, atorvastatin or a pharmaceutically acceptable salt, solvate or hydrate thereof. In a preferred embodiment of the invention, atorvastatin calcium is used.

The concentration of atorvastatin in the composition is preferably between about 0.1 mg/mL and 100 mg/mL, more preferably between about 1 mg/mL and 80 mg/mL and even more preferably between about 2 mg/mL and 40 mg/mL. Specifically, the following concentrations of atorvastatin in the composition are favorable: 0.5 mg/mL, 2 mg/mL, 8 mg/mL, 10 mg/mL, 20 mg/mL, 30 mg/mL and 40 mg/mL.

The composition of the invention is in the form of a suspension, such that solid atorvastatin particles are suspended in the non-polar carrier. The atorvastatin particles in the carrier may be of any size, and typically have a d90 of between about 5 μm and 30 μm, and preferably between about 8 μm to 15 μm, when determined by laser diffraction analysis.

The compositions of the invention further comprise a non-polar carrier. The term ‘non-polar carrier’ refers to carriers which are water-immiscible. Typically, non-polar carriers may be characterized by having a dielectric constant of 15 or less. The non-polar carrier preferably comprises a glyceride, and more preferably a triglyceride or mixture of triglycerides. In a preferred embodiment of the invention, the non-polar carrier comprises medium chain triglyceride. The term ‘medium chain triglyceride’ refers to triglycerides in which at least two of the three fatty acids attached to the glycerol backbone are of medium length, i.e. have a chain length of between six and twelve carbon atoms.

In a specific embodiment, the carrier is selected from a group of medium chain triglycerides which are commercially available as Miglyols. Non-limiting examples of Miglyols that may be employed in the compositions of the invention include Miglyol 612 (Glyceryl Trihexanoate), Miglyol 808 (Tricaprylin), Miglyol 810 (Caprylic/Capric Triglyceride), Miglyol 812 (Caprylic/Capric Triglyceride), Miglyol 818 (Caprylic/Capric/Linoleic Triglyceride), Miglyol 829 (Caprylic/Capric/Succinic Triglyceride), Miglyol 8108 (Caprylic/Capric Triglyceride), Miglyol 840 (Propylene Glycol Dicaprylate/Dicaprate), Miglyol 8810 (Butylene Glycol Dicaprylate/Dicaprate), with Miglyol 812 being especially preferred.

In addition to the atorvastatin and carrier, the compositions of the present invention may optionally further comprise additional components such as sweeteners, flavoring agents and/or thickening agents.

Suitable sweeteners that may be used in the composition of the invention include, for example, acesulfame K, glycamil, neohessperidine dihydrochalone NH, saccharin, saccharin sodium, sucralose, sucrose, thamatin, stevia, aspartame and neotame, with sucralose being especially preferred. The concentration of the sweetener in the composition is typically between 0 and 10% weight, and preferably between 0 and 0.5% weight.

Non-limiting examples of flavoring agents that may be used in the composition of the invention include blackcurrant, strawberry, orange, vanillin, peppermint, raspberry and aniseed flavours, with blackcurrant and orange flavours being especially preferred. The concentration of the flavoring agents in the composition is typically between 0 and 5% weight, and preferably between 0 and 2% weight.

Thickening agents may be added to the atorvastatin compositions. Any pharmaceutically acceptable thickening agents that are compatible with non-polar carriers may be used. In a preferred embodiment, silicon dioxide is used as the thickening agent. The concentration of the thickening agent in the composition is typically between and 50% weight, and preferably between 0 and 20% weight.

In a specific embodiment, the composition of the invention comprises atorvastatin calcium trihydrate in a preferable concentration of 2 mg atorvastatin/mL, suspended in Miglyol 812 N. The composition may optionally further comprise silicon dioxide, preferably in a concentration of 5% weight. The composition may optionally further comprise one or more of sweetener agents (such as sucralose) and flavoring agents (such as orange and/or blackcurrant flavors).

It has been found that after standing for 3 months at an elevated temperature, the compositions of the invention remains stable and contains substantially less degradation products as compared to an analog aqueous atorvastatin suspension composition. Such degradation products include, for example, (3R,5R)-7-[5-(4-fluorophenyl)-3-isopropyl-2-oxo-4-phenyl-3-(phenylcarbomyl)-2,3-dihydro-1H-pyrrol-1-yl]-3,5-dihydroxyheptanoic acid; (4R,6R)-6-[2-[2-(4-fluorophenyl)-5-(1-methylethyl)-3-phenyl-4-(phenylcarbamoyl)-1H-pyrrol-1-yl]ethyl]-4-hydroxytetrahydro-2H-pyran-2-one; 3-[(4-fluorophenyl)carbonyl]-2-(2-methylpropanoyl)-N,3-diphenyloxirane-2-carboxamide; and 4-(4-fluorobenzoyl)-2,4-dihydroxy-2-isopropyl-N,5-diphenyl-3,6-dioxabicyclo [3.1.0] hexane-1-carboxamide.

The composition of the invention can be prepared by mixing together the ingredients. Typically, the mixing is carried out using a high shear mixer.

Accordingly, in another embodiment, the invention provides a process for preparing an atorvastatin composition, comprising mixing atorvastatin, or a pharmaceutically acceptable salt, solvate or hydrate thereof, with at least one non-polar carrier. Preferably, the mixing is carried out until the atorvastatin, or pharmaceutically acceptable salt, solvate or hydrate thereof, is homogeneously dispersed in the non-polar carrier. Optionally, additional components, such as sweeteners, flavoring agents and/or thickening agents, are added to the mixture.

The atorvastatin compositions of the present invention are suitable for oral administration, and may be used to reduce cholesterol levels and/or prevent cardiovascular events in an individual. The suspension compositions are especially suitable for use for the administration of atorvastatin to individuals having difficulty swallowing solid oral dosage forms, such as infants, elders, or other individuals suffering from dysphagia.

Accordingly, in another embodiment, the invention provides a method for the reduction of cholesterol levels and/or prevention of cardiovascular events in an individual, comprising orally administering to said individual the composition of the invention, which comprises atorvastatin and a non-polar carrier, and is in the form of a suspension. Preferably, the non-polar carrier is Miglyol, more preferably Miglyol 812.

The invention is further illustrated by the following examples, which are not to be construed as limiting.

### EXAMPLES

All percentages herein are weight percentages unless otherwise indicated.

Where used herein, the term “room temperature” refers to a temperature in the range from about 20° C. to 30° C., such as, for example, 25° C.

Methods

Stability tests were carried out using HPLC (Thermo Scientific Ultimate 3000) with a Synergi Polar RP analytical column (4μ 250×4.6 mm).

The following eluent system (shown in Table 1) was used as a mobile phase:

TABLE 1

| Buffer | 0.63 g/L ammonium | | | |
|---|---|---|---|---|
| | formate, adjusted to | | | |
| | pH 3.5 with formic acid | | | |
| | solution | | | |
| Mobile phase: A | Acetonitrile | | | |
| Mobile phase: B | 40:60 | | | |
| | Acetonitrile:Buffer | | | |
| Program File: | | | | |
| Gradient Step | Time | % A | % B | |
| 1 | 0.0 | 0.0 | 100.0 | |
| 2 | 22.0 | 12.0 | 88.0 | |
| 3 | 27.0 | 75.0 | 25.0 | |
| 4 | 29.4 | 75.0 | 25.0 | |
| 5 | 29.5 | 100.0 | 0.0 | To remove oil from |
| 6 | 30.5 | 100.0 | 0.0 | system. (Not required |
| | | | | for aqueous suspensions) |
| 7 | 30.6 | 0.0 | 100.0 | |
| 8 | 35.0 | 0.0 | 100.0 | |

| | |
|---|---|
| Flow Rate: | 1.5 mL/min. |
| Injection Volume: | 7.5 μl. |
| Oven Temperature: | 30° C. |
| UV Wavelength: | 248 nm |
| Autosampler Temp: | 5° C. |
| Run Time: | 35.0 mins |
| Needle Wash/ | 50:50 |
| Column Wash: | Acetonitrile:Water |

### Definitions

The following abbreviations are used in the examples:

ND: not detected.

RT: retention time.

RRT: relative retention time (relative to the main peak).

URRT: unspecified (degradation product) relative retention time.

OWS: off-white suspension.
oxo impurity: (3R,5R)-7-[5-(4-Fluorophenyl)-3-isopropyl-2-oxo-4-phenyl-3-(phenylcarbomyl)-2,3-dihydro-1H-pyrrol-1-yl]-3,5-dihydroxyheptanoic acid.
impurity H (lactone): (4R,6R)-6-[2-[2-(4-fluorophenyl)-5-(1-methylethyl)-3-phenyl-4-(phenylcarbamoyl)-1H-pyrrol-1-yl]ethyl]-4-hydroxytetrahydro-2H-pyran-2-one.
impurity D: 3-[(4-fluorophenyl)carbonyl]-2-(2-methylpropanoyl)-N,3-diphenyloxirane-2-carboxamide.
impurity D (ETHFA): 4-(4-fluorobenzoyl)-2,4-dihydroxy-2-isopropyl-N,5-diphenyl-3,6-dioxabicyclo[3.1.0] hexane-1-carboxamide.

The latter is a cyclic hemiketal of atorvastatin impurity D, atorvastatin epoxy tetrahydrofuran analog (ETHFA).

### Comparative Preparation 1

### Preparation of an Aqueous Atorvastatin Suspension

In a first vessel, microcrystalline cellulose (Avicel; 14.00 g) was added to 1000.0 g purified water. Citric acid monohydrate (0.60 g) was added and mixed until dissolution. Disodium hydrogen phosphate dihydrate (9.40 g) was added and mixed until dissolution. Simethicone emulsion (Q7 2587 30%; 1.00 g) was added and mixed until fully dispersed. Polysorbate 80 (1.00 g) was added and mixed until homogeneous.

In a second vessel, sodium methyl hydroxybenzoate (4.177 g) and sodium ethyl hydroxybenzoate (2.135 g) were added to 200.0 g purified water and mixed until full dissolution. The solution was then added to the first vessel and mixed until homogeneous. Sucralose (4.00 g) was added and mixed until dissolution. Orange flavor (4.00 g) and blackcurrant flavor (1.00 g) were added and mixed until homogeneous. Atorvastatin calcium trihydrate (4.542 g; having a water content of 4.95% w/w) was added and mixed until fully dispersed. carboxymethylcellulose sodium (4.00 g) was added and mixed with a high shear mixer until homogeneous. Glycerol (400.0 g) was added and mixed until homogeneous. The mixture was then split equally between two separate vessels, A and B.

Aqueous Suspension 1A (2 mg/mL)—

The pH of the mixture in vessel A was adjusted to 8.0 by the addition of a sodium hydroxide solution and/or a hydrochloric acid solution as needed. Purified water was added to vessel A to adjust the final volume of the mixture to 1.0 L.

Aqueous Suspension 1B (2 mg/mL)—

The pH of the mixture in vessel B was adjusted to 7.0 by the addition of a sodium hydroxide solution and/or a hydrochloric acid solution as needed. Purified water was added to vessel B to adjust the final volume of the mixture to 1.0 L.

### Example 1

### Preparation of Oil-Based Atorvastatin Suspensions

Oil Suspension 1 (2 mg/mL)

In a vessel, atorvastatin calcium trihydrate (2.26 g; having a water content of 4.4% w/w) was added to medium chain triglyceride (miglyol 812 N; 400.0 g) and mixed until fully dispersed using a high shear mixer. Sucralose (1.00 g), orange flavor (Flavex; 1.00 g) and blackcurrant flavor (0.25 g) were added and mixed until dissolution using a high shear mixer. Miglyol 812 N was added to a final volume of 1000 mL.

Oil Suspensions 2A-2E (8 mg/mL)

In a vessel, colloidal silicon dioxide (Aerosil 200; 100.0 g) was added to medium chain triglyceride (miglyol 812 N; 2000.0 g) and mixed until dissolved using a high shear mixer. Atorvastatin calcium trihydrate (45.3 g; having a water content of 4.4% w/w) was added and mixed until fully dispersed using a high shear mixer. The mixture was then split equally between five separate vessels, A-E.

Oil Suspension 2A—

Miglyol 812 N was added to vessel A to a final volume of 1000 mL and the formulation mixed until fully homogeneous using a high shear mixer.

Oil Suspension 2B—

Sucralose (1.00 g) was added to vessel B and mixed until dissolution. Miglyol 812 N was added to a final volume of 1000 mL and the formulation mixed until fully homogeneous using a high shear mixer.

Oil Suspension 2C—

Sucralose (1.00 g) was added to vessel C and mixed until dissolution. Orange flavor (Flavex; 1.00 g) was added and mixed until homogeneous using a high shear mixer. Miglyol 812 N was added to a final volume of 1000 mL and the formulation mixed until fully homogeneous using a high shear mixer.

Oil Suspension 2D—

Sucralose (1.00 g) was added to vessel D and mixed until dissolution. Blackcurrant flavor (0.25 g) was added and mixed until homogeneous using a high shear mixer. Miglyol 812 N was added to a final volume of 1000 mL and the formulation mixed until fully homogeneous using a high shear mixer.

Oil Suspension 2E—

Sucralose (1.00 g) was added to vessel E and mixed until dissolution. Orange flavor (Flavex; 1.00 g) and blackcurrant flavor (0.25 g) were added and mixed until homogeneous using a high shear mixer. Miglyol 812 N was added to a final volume of 1000 mL and the formulation mixed until fully homogeneous using a high shear mixer.

Oil Suspensions 3-7

Oil suspensions 3 (0.5 mg/mL atorvastatin), 4 (10 mg/mL atorvastatin), 5 (20 mg/mL atorvastatin), 6 (30 mg/mL atorvastatin) and 7 (40 mg/mL atorvastatin) were prepared. The ingredients of oil suspensions 3-7 are described in Table 2 below. The oil suspensions were prepared by the following method:

In a vessel, atorvastatin calcium trihydrate was added to medium chain triglyceride (miglyol 812 N) and mixed until fully dispersed using a high shear mixer. Sucralose, orange flavor and blackcurrant flavor were added and mixed using a high shear mixer. Miglyol 812 N was added to a final volume of 500 mL and mixed using a high shear mixer.

TABLE 1

| | Oil suspension # | | | | |
|---|---|---|---|---|---|
| Ingredient | 3 | 4 | 5 | 6 | 7 |
| Miglyol 812 N | 400.0 g | 400.0 g | 400.0 g | 400.0 g | 400.0 g |
| Atorvastatin | 0.28 g | 5.67 g | 11.35 g | 17.0 g | 22.7 g |
| calcium trihydrate | | | | | |
| (water content | | | | | |
| 4.6% w/w) | | | | | |
| Sucralose | 0.50 g | 0.50 g | 0.50 g | 0.50 g | 0.50 g |
| Orange flavor | 0.50 g | 0.50 g | 0.50 g | 0.50 g | 0.50 g |
| (Flavex) | | | | | |
| Blackcurrant | 0.125 g | 0.125 g | 0.125 g | 0.125 g | 0.125 g |
| flavour | | | | | |
| Miglyol 812 N | To 500 mL | To 500 mL | To 500 mL | To 500 mL | To 500 mL |

### Example 2

### Stability Tests—2 mg/mL Atorvastatin Oil Suspension 1

Atorvastatin ‘oil suspension 1’ (prepared in example 1) was kept in storage at two different temperatures (25° C. and 30° C.) for 9 months. Samples of the suspensions were analyzed for degradation products by HPLC after 0, 1, 3, and 9 months of storage. The results are shown in Tables 3 and 4 below.

TABLE 3
Test 1- 25° C.

| | | | Atorvastatin | | |
|---|---|---|---|---|---|
| | | | (% of stated | | |
| Time | | | amount = % | Oxo | Impurity H |
| Point | | Density | of 2 mg/mL) | Impurity | (Lactone) |
| (months) | Description | (g/mL) | RT = 18.3 mins | RRT = 0.88* | RRT = 1.42* |
| 0 | OWS+ | 0.947 | 102.95 | ND | ND |
| 1 | OWS+ | 0.947 | 101.00 | <0.05 | <0.05 |
| 3 | OWS+ | 0.947 | 101.07 | 0.06 | 0.05 |
| 6 | OWS+ | 0.947 | 100.71 | 0.08 | 0.06 |
| 9 | OWS+ | 0.947 | 100.09 | 0.10 | 0.06 |

| | | | | | Total |
|---|---|---|---|---|---|
| Time | Impurity D | | | | Degradation |
| Point | (ETHFA) | Impurity D | URRT | URRT | Products |
| (months) | (RRT = 1.49)* | (RRT = 1.52)* | 1.41* | 1.43* | (% weight)** |
| 0 | ND | ND | ND | ND | 0.00 |
| 1 | <0.05 | <0.05 | ND | ND | 0.00 |
| 3 | <0.05 | <0.05 | <0.05 | <0.05 | 0.00 |
| 6 | <0.05 | <0.05 | <0.05 | <0.06 | 0.00 |
| 9 | 0.05 | 0.05 | 0.05 | 0.07 | 0.10 |

TABLE 4
Test 2- 30° C.

| | | | Atorvastatin | | |
|---|---|---|---|---|---|
| | | | (% of stated | | |
| Time | | | amounts = % | Oxo | Impurity H |
| Point | | Density | of 2 mg/mL) | Impurity | (Lactone) |
| (months) | Description | (g/mL) | RT = 18.3 mins | (RRT 0.88)* | (RRT 1.42)* |
| 0 | OWS+ | 0.947 | 102.95 | ND | ND |
| 1 | OWS+ | 0.947 | 101.52 | <0.05 | 0.05 |
| 3 | OWS+ | 0.947 | 100.86 | 0.07 | 0.06 |
| 6 | OWS+ | 0.947 | 100.86 | 0.10 | 0.07 |
| 9 | OWS+ | 0.947 | 99.89 | 0.12 | 0.08 |

| | | | | | Total |
|---|---|---|---|---|---|
| Time | Impurity D | | | | Degradation |
| Point | (ETHFA) | Impurity D* | URRT | URRT | Products |
| (months) | (RRT 1.49)* | (RRT 1.52)* | 1.41* | 1.43* | (% weight)** |
| 0 | ND | ND | ND | ND | 0.00 |
| 1 | <0.05 | <0.05 | ND | ND | 0.00 |
| 3 | <0.05 | <0.05 | <0.05 | <0.05 | 0.00 |
| 6 | 0.05 | <0.05 | 0.05 | 0.07 | 0.10 |
| 9 | 0.06 | 0.05 | 0.06 | 0.09 | 0.12 |

+ Exhibits sedimentation at the bottom of the flask and at the top of the flask. The sedimentation is easily re-suspended upon shaking.

* % relative to nominal atorvastatin amount.

** Only peaks above reporting threshold (0.1%) are included in total.

### Example 3 (Comparative)

### Stability Tests—2 mg/mL Atorvastatin Aqueous Suspensions

Atorvastatin aqueous suspensions 1A and 1B (prepared in comparative preparation 1) were kept in storage at two different temperatures (5° C. and 25° C.) for 2 months. Samples of the suspensions were analyzed for degradation products by HPLC after 0, 1 and 2 months of storage. The results are shown in Tables 5 and 6 below.

TABLE 5
Test 1- 5° C.

| | | | | | Atorvastatin | Methyl | Ethyl |
|---|---|---|---|---|---|---|---|
| | | | | | (% of stated | Hydroxybenzoate | Hydroxybenzoate |
| Aqueous | Time | | | | amount = % | (% of stated | (% of stated |
| suspension | Point | | Density | | of 2 mg/mL) | amount = % | amount = % |
| # | (months) | Description | (g/mL) | pH | RT = 18.3 mins | of 1.8 mg/mL) | of 0.9 mg/mL) |
| 1A | 0 | OWS | 1.054 | 8.04 | 100.46 | 100.29 | 96.93 |
| 1A | 1 | OWS | 1.051 | 8.00 | 99.15 | 98.51 | 96.00 |
| 1A | 2 | OWS | 1.051 | 7.90 | 100.15 | 100.11 | 95.77 |
| 1B | 0 | OWS | 1.054 | 7.11 | 99.98 | 99.70 | 96.28 |
| 1B | 1 | OWS | 1.052 | 7.08 | 98.12 | 98.89 | 95.63 |
| 1B | 2 | OWS | 1.052 | 7.05 | 98.43 | 101.26 | 95.62 |

| | | Oxo | Impurity H | Impurity D | | | | Total |
|---|---|---|---|---|---|---|---|---|
| Aqueous | Time | Impurity | (Lactone) | (ETHFA) | Impurity D | | | degradation |
| suspension | Point | (RRT | (RRT | (RRT | (RRT | URRT | URRT | products |
| # | (months) | 0.88)* | 1.42)* | 1.49)* | 1.52)* | 0.74* | 1.20* | (% weight) ** |
| 1A | 0 | ND | ND | 0.13 | ND | ND | ND | 0.13 |
| 1A | 1 | 0.11 | 0.05 | 0.18 | <0.05 | ND | ND | 0.29 |
| 1A | 2 | 0.49 | 0.05 | 0.19 | <0.05 | ND | ND | 0.68 |
| 1B | 0 | ND | ND | 0.15 | ND | ND | ND | 0.15 |
| 1B | 1 | 0.12 | 0.20 | 0.20 | <0.05 | ND | ND | 0.52 |
| 1B | 2 | 0.49 | 0.40 | 0.23 | <0.05 | ND | ND | 1.12 |

TABLE 6
Test 2- 25° C.

| | | | | | Atorvastatin | Methyl | Ethyl |
|---|---|---|---|---|---|---|---|
| | | | | | (% of stated | Hydroxybenzoate | Hydroxybenzoate |
| Aqueous | Time | | | | amount = % | (% of stated | (% of stated |
| suspension | Point | | Density | | of 2 mg/mL) | amount = % of | amount = % of |
| # | (months) | Description | (g/mL) | pH | RT = 18.3 mins | 1.8 mg/mL) | 0.9 mg/mL) |
| 1A | 0 | OWS | 1.054 | 8.04 | 100.46 | 100.29 | 96.93 |
| 1A | 1 | OWS | 1.052 | 7.93 | 98.53 | 91.15 | 93.21 |
| 1A | 2 | OWS | 1.052 | 7.87 | 98.57 | 83.39 | 89.69 |
| 1B | 0 | OWS | 1.054 | 7.11 | 99.98 | 99.70 | 96.28 |
| 1B | 1 | OWS | 1.052 | 7.05 | 97.24 | 97.38 | 95.14 |
| 1B | 2 | OWS | 1.052 | 6.97 | 97.50 | 97.86 | 94.65 |

| | | Oxo | Impurity H | Impurity D | | | | Total |
|---|---|---|---|---|---|---|---|---|
| | Time | Impurity | (Lactone) | (ETHFA) | Impurity D | | | degradation |
| Aqueous | Point | (RRT | (RRT | (RRT | (RRT | URRT | URRT | products |
| suspension # | (months) | 0.88)* | 1.42)* | 1.49)* | 1.52)* | 0.74* | 1.20* | (% weight)** |
| 1A | 0 | ND | ND | 0.15 | ND | ND | ND | 0.13 |
| 1A | 1 | 0.43 | 0.07 | 0.29 | <0.05 | 0.11 | ND | 0.71 |
| 1A | 2 | 2.16 | 0.08 | 0.16 | <0.05 | 0.22 | 0.11 | 2.65 |
| 1B | 0 | ND | ND | 0.15 | ND | ND | ND | 0.15 |
| 1B | 1 | 0.48 | 0.46 | 0.29 | <0.05 | ND | ND | 1.23 |
| 1B | 2 | 2.45 | 0.51 | 0.36 | <0.05 | 0.15 | ND | 3.47 |

*% relative to nominal atorvastatin amount
** Only peaks above reporting threshold (0.1%) are to be included in total.

1. A composition comprising atorvastatin, or a pharmaceutically acceptable salt, solvate or hydrate thereof, and a non-polar carrier, wherein said composition is in the form of a suspension.

2. A composition comprising atorvastatin, or a pharmaceutically acceptable salt, solvate or hydrate thereof, and a non-polar carrier, wherein said atorvastatin or salt thereof is suspended in the composition.

3. The composition of claim 1, wherein the non-polar carrier comprises a triglyceride.

4. The composition of claim 3, wherein the triglyceride is Miglyol.

5. The composition of claim 4, wherein the Miglyol is 812.

6. The composition of claim 1, wherein the composition further comprises one or more components selected from the group consisting of sweeteners, flavoring agents, thickening agents and preservatives.

7. The composition of claim 1 which comprises atorvastatin calcium, or a hydrate thereof, and Miglyol 812 N as the non-polar solvent.

8. The composition of claim 7, wherein the atorvastatin calcium is in a concentration of between 0.5 and 40 mg atorvastatin/mL.

9. The composition of claim 8, which further comprises silicon dioxide in a concentration of 5% weight.

10. A method for the reduction of cholesterol levels in an individual, comprising orally administering to said individual the composite of claim 1.

11. The composition of claim 2, wherein the non-polar carrier comprises a triglyceride.

12. The composition of claim 11, wherein the triglyceride is Miglyol.

13. The composition of claim 12, wherein the Miglyol is 812.

14. The composition of any of claim 2, wherein the composition further comprises one or more components selected from the group consisting of sweeteners, flavoring agents, thickening agents and preservatives.

15. The composition of claim 2 which comprises atorvastatin calcium, or a hydrate thereof, and Miglyol 812 N as the non-polar solvent.

16. The composition of claim 15, wherein the atorvastatin calcium is in a concentration of between 0.5 and 40 mg atorvastatin/mL.

17. The composition of claim 16, which further comprises silicon dioxide in a concentration of 5% weight.
